(19) United States
(12) Patent Application Publication
(10) Pub. No.: US 2025/0132050 A1
(43) Pub. Date: Apr. 24, 2025

(54) SYSTEMS AND METHODS FOR DISPLAYING HEALTH METRICS

(71) Applicant: Hanger, Inc., Austin, TX (US)

(72) Inventors: Zlatko Larry Hodin, Reno, NV (US); Barry Thomson, Reno, NV (US)

(73) Assignee: Hanger, Inc., Austin, TX (US)

(21) Appl. No.: 18/922,919

(22) Filed: Oct. 22, 2024

Related U.S. Application Data
(60) Provisional application No. 63/592,420, filed on Oct. 23, 2023.

(51) Int. Cl. G16H 50/30

(52) U.S. Cl. CPC G16H 50/30

(57) ABSTRACT

A system for scoring and displaying a health metric of a first user includes processing circuitry configured to provide a gamified prompt to the first user via a display. The processing circuitry is further configured to obtain a first measurement of the health metric of the first user from a measurement device. The measurement device configured to obtain the first measurement of the health metric of the first user. The processing circuitry is further configured to generate a first normalized score by normalizing the first measurement of the health metric of the first user relative to a plurality of measurements of the health metric of a first population of individuals. The first user is a member of the first population of individuals. The processing circuitry is further configured to provide the first normalized score to the display as game results.

### CROSS-REFERENCE TO RELATED PATENT APPLICATION

This application claims the benefit of and priority to U.S. Provisional Patent Application No. 63/592,420, filed Oct. 23, 2023, which is incorporated herein by reference in its entirety.

### BACKGROUND

The present disclosure relates to obtaining health metrics. More specifically, the present disclosure relates to displaying health metrics of a patient.

### SUMMARY

One embodiment relates to a system for scoring and displaying a health metric of a first user. The system includes processing circuitry configured to provide a gamified prompt to the first user via a display, obtain a first measurement of the health metric of the first user from a measurement device, generate a first normalized score by normalizing the first measurement of the health metric of the first user relative to a plurality of measurements of the health metric of a first population of individuals, and provide the first normalized score to the display as game results. The measurement device is configured to obtain the first measurement of the health metric of the first user. The first user is a member of the first population of individuals.

In some embodiments, a second user is a member of a second population of individuals. The processing circuitry is further configured to generate a second normalized score by normalizing a second measurement of the health metric of the second user relative to a plurality of measurements of the health metric of the second population of individuals and provide the second normalized score to the display as game results. In some embodiments, the measurement device is a spirometer device, and the health metric is a metric indicating lung health. In some embodiments, the gamified prompt is a prompt for the first user to perform an action in order to achieve a game objective, wherein the measurement device obtains the first measurement of the health metric of the first user as the first user performs the action. In some embodiments, the processing circuitry is configured to provide the first normalized score to the display as a result of the action performed in order to achieve the game objective.

In some embodiments, the game results are positive when the first normalized score exceeds a threshold score, the threshold score corresponding with health metrics that are determined to be healthy. In some embodiments, the game results include a game element that is operated to at least one of move at a speed based on the first normalized score, raise to a height based on the first normalized score, or move a distance based on the first normalized score. In some embodiments, the first population of individuals is based on at least one of age or sex.

Another embodiment relates to a method for scoring and displaying a health metric of a first user. The method includes obtaining a first measurement of the health metric of a first user from a measurement device, generating a first normalized score by normalizing the first measurement of the health metric of the first user relative to a plurality of measurements of the health metric of a first population of individuals, and providing the first normalized score as game results to the first user via a display. The measurement device is configured to obtain the first measurement of the health metric of the first user. The first user is a member of the first population of individuals.

In some embodiments, the method further includes obtaining a second measurement of the health metric of a second user from the measurement device, generating a second normalized score by normalizing the second measurement of the health metric of the second user relative to a plurality of measurements of the health metric of a second population of individuals, and providing the second normalized score to the display as game results. The second user is a member of the second population of individuals. In some embodiments, the first normalized score and the second normalized score are simultaneously displayed on the display as game results. In some embodiments, the first population of individuals and the second population of individuals are based on at least one of age or sex. In some embodiments, the measurement device is a spirometer device, and the health metric is a metric indicating lung health. In some embodiments, the game results are positive when the first normalized score exceeds a threshold score, the threshold score corresponding with health metrics that are determined to be healthy.

In some embodiments, the method further includes obtaining a second measurement of the health metric of the first user from the measurement device, generating a second normalized score by normalizing the second measurement of the health metric of the first user relative to the plurality of measurements of the health metric of the first population of individuals, and providing the second normalized score as game results to the display. In some embodiments, the first normalized score and the second normalized score are simultaneously displayed on the display as game results.

Yet another embodiment relates to a non-transitory computer-readable media having computer-executable instructions embodied therein that, when executed by at least one processor, causes the at least one processor to obtain a first measurement of a health metric of a first user from a measurement device, obtain, from a database, a plurality of measurements of the health metric of a first population of individuals, generate a first normalized score by normalizing the first measurement of the health metric of the first user relative to the plurality of measurements of the health metric of the first population of individuals, and provide the first normalized score to a display as game results. The first user is a member of the first population of individuals.

In some embodiments, the instructions further cause the at least one processor to generate a second normalized score by normalizing a second measurement of the health metric of a second user relative to a plurality of measurements of the health metric of a second population of individuals and provide the second normalized score to the display as game results. The second user is a member of the second population of individuals. In some embodiments, the instructions further cause the at least one processor to obtain a third measurement of the health metric of the first user from the measurement device, generate a third normalized score by normalizing the third measurement of the health metric of the first user relative to the plurality of measurements of the health metric of the first population of individuals, and provide the third normalized score as game results to the display. In some embodiments, the measurement device is a spirometer device, and the health metric is a metric indicating lung health.

This summary is illustrative only and is not intended to be in any way limiting. Other aspects, inventive features, and advantages of the systems, devices, or processes described herein will become apparent in the detailed description set forth herein, taken in conjunction with the accompanying figures, wherein like reference numerals refer to like elements. Numerous specific details are provided to impart a thorough understanding of embodiments of the subject matter of the present disclosure. The described features of the subject matter of the present disclosure may be combined in any suitable manner in one or more embodiments and/or implementations. In this regard, one or more features of an aspect of the invention may be combined with one or more features of a different aspect of the invention. Moreover, additional features may be recognized in certain embodiments and/or implementations that may not be present in all embodiments or implementations.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a block diagram of a computing environment that provides a gamified health metric of a user, according to some embodiments.

FIG. 2 is flow diagram of a method for providing a gamified health metric to a user, according to some embodiments.

FIG. 3 is a graphical user interface providing a gamified prompt to a user, according to some embodiments.

FIG. 4 is a graphical user interface providing a gamified health metric to a user, according to some embodiments.

FIG. 5 is a graphical user interface providing a gamified health metric to a user, according to some embodiments.

FIG. 6 is a graphical user interface providing a gamified health metric to a user, according to some embodiments.

FIG. 7 is a graphical user interface providing a gamified health metric to a user, according to some embodiments.

FIG. 8 is a graphical user interface providing a gamified health metric to a user, according to some embodiments.

FIG. 9 is a graphical user interface providing a gamified health metric to a user, according to some embodiments.

FIG. 10 is a graphical user interface providing a gamified health metric to a user, according to some embodiments.

FIG. 11 is a graphical user interface providing a gamified health metric to a user, according to some embodiments.

FIG. 12 is a graphical user interface providing a gamified health metric to a user, according to some embodiments.

FIG. 13 is a graphical user interface providing a gamified health metric to a user, according to some embodiments.

FIG. 14 is a graphical user interface providing a gamified health metric to a user, according to some embodiments.

FIG. 15 is a graphical user interface providing a gamified health metric to a user, according to some embodiments.

FIG. 16 is a graphical user interface providing a gamified health metric to a user, according to some embodiments.

### DETAILED DESCRIPTION

Referring generally to the FIGURES, systems and methods for providing normalized scores of measurements of a health metric as game results are disclosed according to various embodiments herein. In some embodiments, the systems and methods provide multiple normalized scores of multiple measurements of a health metric for different users as game results where the different users belong to different demographics and the measurements of each of the users is normalized to measurements from each of the demographics of the users respectively. For example, a first measurement of a health metric of a parent and a second health metric of a child may be normalized to measurements of individuals within the respective age groups of the parent and the child and displayed as game results such that the child and the parent may compete over the game results despite a vast difference in the measurements of the parent and the child. Additionally, in some embodiments, the system and methods provide normalized scores of measurements of lung heath as game results and the measurements are taken using a spirometer device.

In some embodiments, the systems and methods described herein include providing a normalized score of a measurement of a health metric of a user as game results. The normalized score may be normalized with measurements of a population of the user. In some embodiments, the systems and method include providing a historical normalized score of a historical measurement of the health metric of the user as game results that is normalized with measurements of a second population that the user was a part of when the historical measurement was taken. For example, in some embodiments, a user may compare the normalized score to the historical score in the game results to determine how the measurements of the health metric of the user have changed relative to the populations of the user over time.

Accordingly, the systems and methods described herein provide a variety of improvements to systems for displaying health metrics. For example, the systems and method described herein may provide users with normalized scores of measurements of health metrics as game scores. As such, users of different demographics may be able to fairly compete over game results of measurements of health metrics due to different demographics having vastly different ranges of the measurements of the health metrics. Further, the systems and methods may allow the users to compare the game results of the multiple users on a display either in real-time time or in a delayed manner (e.g., a first patient plays the game at a first time and a second patient plays the game at a second time).

The systems and methods described herein may provide users with normalized scores of measurements of a health metric as game results. As such, users may easily compare game results associated with the measurements of the health metric between users of different demographics without certain demographics having an advantage in the game results over other demographics.

Before turning to the figures, which illustrate certain example embodiments in detail, it should be understood that the present disclosure is not limited to the details or methodology set forth in the description or illustrated in the figures. It should also be understood that the terminology used herein is for the purpose of description only and should not be regarded as limiting.

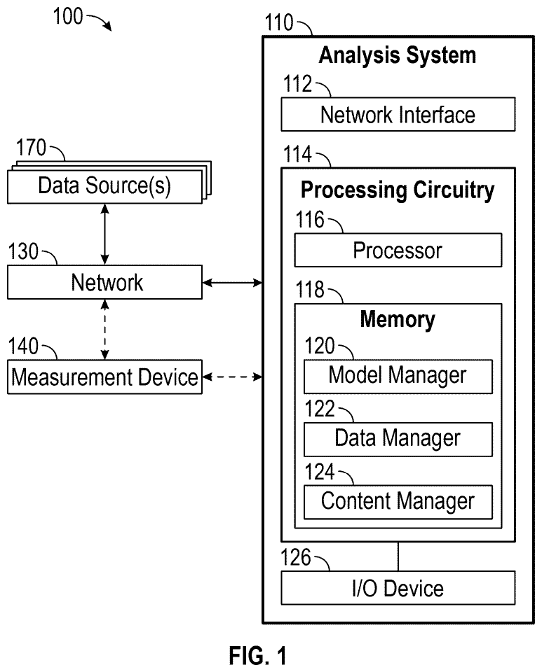

Referring to FIG. 1, a block diagram of a computing environment 100 including an analysis system 110 is shown, according to some embodiments. The analysis system 110 is configured to receive data from a measurement device 140 (e.g., a medical measurement device such as a spirometer device, a grip strength trainer, or another device that takes measurements of a patient), according to some embodiments. In some embodiments, the analysis system 110 is associated with a provider, such as a health care provider, a hospital, a physical therapist, an occupational therapist, a respiratory therapist, a nurse, a speech pathologist, or a language pathologist. The computing environment 100 may include one or more measurement devices 140 (e.g., multiple of a same type of the measurement device 140, different types of the measurement device 140, etc.), and one or more data sources (e.g., data source 170). In some embodiments, the analysis system 110, measurement device 140 (as well as any additional measurement devices), and data source 170 are communicatively coupled (e.g., wiredly, wirelessly, via a local network, via the Internet, etc.). The various components of the computing environment are in communication with each other and are connected by a network 130, according to some embodiments.

Although the various systems and devices are shown in FIG. 1 as being singular, it will be understood that, in some embodiments, the computing environment 100 includes one or multiple of any of the various illustrated systems and/or devices, as desired for a given application. Similarly, while the following descriptions of the various systems and devices are largely provided in terms of single systems or devices, it will be appreciated that these descriptions are similarly applicable to any additional corresponding systems and/or devices (e.g., additional of the data sources 170, additional of the measurement devices 140, and so on).

The analysis system 110 may be operated by a provider, such as an entity, a healthcare provider, a physical therapist, a medical professional, etc. The analysis system 110 includes a network interface 112 that connects the analysis system 110 with the network 130, according to some embodiments. The network interface 112 facilitates secure communications between the analysis system 110 and various other components of the computing environment 100, according to some embodiments. Further, in some embodiments, the network interface 112 includes cryptography capabilities to establish a secure or relatively secure communication session in which data communicated over the session is encrypted.

The analysis system 110 includes processing circuitry 114 having a processor 116 and memory 118, according to some embodiments. The processing circuitry 114 may be implemented as one or more application specific integrated circuits (ASICs), field programmable gate arrays (FPGAs), a group of processing components, or other suitable electronic processing components. The processor 116 may be implemented as one or more application specific integrated circuits (ASICs), field programmable gate arrays (FPGAs), a group of processing components, or other suitable electronic processing components. The memory 118 may be a device for storing data and/or computer code. In some embodiments, memory 118 may be any volatile or non-volatile computer-readable storage medium capable of storing data or computer code relating to the activities described herein. According to some embodiments, memory 118 includes computer code modules (e.g., executable code, object code, source code, script code, machine code, etc.) configured for execution by processing circuitry 114 on processor 116. The memory 118 may store data associated with a variety of application programs ran by the analysis system 110. In some embodiments, the processor 116 and the memory 118 are communicably coupled with each other via the processing circuitry 114 for implementing one or more of the processes described herein.

The memory 118 includes a model manager 120, a data manager 122 (e.g., a database, etc.), and a content manager 124, according to some embodiments. The model manager 120 is structured or configured to perform a variety of the functionalities described herein. In some embodiments, the model manager 120 is configured to receive a measurement of a health metric of a user from a measurement device and generate a normalized game score (e.g., a first normalized game score, a normalized score, etc.) and game results associated with the normalized game score. In some embodiments, the model manager 120 is further configured to receive a real time measurement of the health metric of the user and generate the normalized game score and game results associated with the normalized game score. The model manager 120 may update the normalized game score and the game results as the model manager 120 receives additional of the real time measurements of the health metric of the user. In some embodiments, the model manager 120 is configured to determine or receive an identification that the user is a member of a population of individuals (e.g., a population demographic, etc.).

The data manager 122 is configured to fuse data, including operations to generate various data structures stored in the memory 118 and used by the analysis system 110 described herein. The data manager 122 may be configured to receive data from multiple sources (e.g., the data sources 170, the measurement devices 140, etc.) and aggregate the data into various data structures stored in the memory 118.

The content manager 124 is configured to generate content for displaying to users via an input-output (“IO”) device 126, according to some embodiments. The content may be selected from various resources (e.g., a request for measurements of a population of individuals from the data manager 122). The content manager 124 may be structured to provide content (e.g., via a graphical user interface (GUI)) to an IO device 126, for display. The content may include actionable items, prompts, or requests that the user may select or otherwise manipulate via the IO device 126. The content may be selected from various resources (e.g., from the data manager 122, from the memory 118, etc.). The IO device 126 may be a display screen, a touch screen, a collection of input and output devices, buttons, a user interface device, a computing device, a smartphone, a tablet, a game system, etc.

The content generated by the content manager 124 may include customized dashboards, such as those described in detail below, with reference to FIGS. 3-5. The content manager 124 may generate customized user-interactive dashboards for one or more entities, such as the IO device 126, based on any of data received from the measurement device 140, data source 170, or any other computing device described therein. The generated dashboards may include various data (e.g., data stored in the content manager 124) associated with one or more measurements including any of measurements of health metrics, normalized measurements of health metrics, measurements of health metrics of a population, game data, normalized game scores, game result data or others. In some embodiments, the analysis system 110 includes an application programming interface (API) and/or a software development kit (SDK) that facilitate the integration of other applications with the analysis system 110. For example, the analysis system 110 is configured to utilize the functionality of the measurement device 140 interacting through an API.

The content manager 124 may generate an interface corresponding to the normalized game score (e.g., generated by the model manager 120). In some embodiments, the interface may include elements illustrating game results including the normalized game scores. The elements may include game elements that move at different speeds depending on the normalized game scores, game elements that raise to different heights or positions depending on the normalized game scores, game elements that move different distances depending on the normalized games scores, or game elements that are otherwise modified or relocated depending on the normalized game scores. For example, the interface may include a first game element that is associated with a first normalized game score and a second game element that is associated with a second normalized game score. If the first normalized game score is higher than the second normalized game score, the first game element may travel at a faster speed than the second game element.

The IO device 126 is structured to receive communications from and provide communications to users associated with the analysis system 110. The IO device 126 may be structured to exchange data, communications, instructions, etc. with an IO component of the analysis system 110 (e.g., a mouse, a monitor, a keyboard, etc.). As such, the IO device 126 may provide an interface for the user to interact with various applications stored on the analysis system 110. The content manager 124 may display content to the users through the IO device 126.

The measurement device 140 is a device that is configured to obtain (e.g., measure, detect, sense, etc.) health related measurements of a patient, according to some embodiments. In some embodiments, the measurement device 140 measures various parameters of a patient and/or collects health-related data of a patient. Data obtained by the measurement device 140 may assist in diagnosing, monitoring, or managing the patient's health. In some embodiments, the measurement device may be or include a spirometer, a blood pressure monitor, a scale, a grip strength tester, and/or any other suitable device for making health related measurements. In some embodiments, the measurement device 140 may include a display for displaying the measurement. The measurement device 140 may communicate with the analysis system 110 through the network 130 (e.g., wirelessly, through WIFI, through Bluetooth, etc.), through a wired connection (e.g., USB, ethernet cables, etc.), or through other means. In some embodiments, the measurement from the measurement device 140 must be entered into the analysis system 110 by hand (e.g., a person reading the measurement from the measurement device 140 and entering the measurement into the analysis system 110, etc.). In some embodiments, the measurement device 140 may include a memory for receiving and storing measurement data of the measurements. Additionally, the measurement devices 140 may each store, in the memory, and execute (“run”) measurement applications, such as running an application that prompts a patient to perform an action and collects the measurements that result from the action.

The data sources 170 may provide data to the analysis system 110 and/or measurement device 140. In some arrangements, the data sources 170 may be structured to collect data from other devices on network 130 (e.g., measurement devices 140 and/or other third-party devices or systems) and relay the collected data to the analysis system 110 and/or measurement device 140. In some embodiments, the analysis system 110 may request data associated with specific data stored in the data source (e.g., data sources 170). For example, in some arrangements, the data sources 170 may support a search or discovery engine for Internet-connected devices. The search or discovery engine may provide data from other providers that, when used to gather measurements of a health metric of a population of individuals, may be used by the model manager 120 to generate a normalized score by normalizing a measurement of the health metric of a user to the measurements of the health metric of the population of individuals. In some embodiments, the data sources 170 may include data relating to health metrics of a population from sources including government health departments, survey data, healthcare provider data, research data, or other sources that gather data related to health metrics.

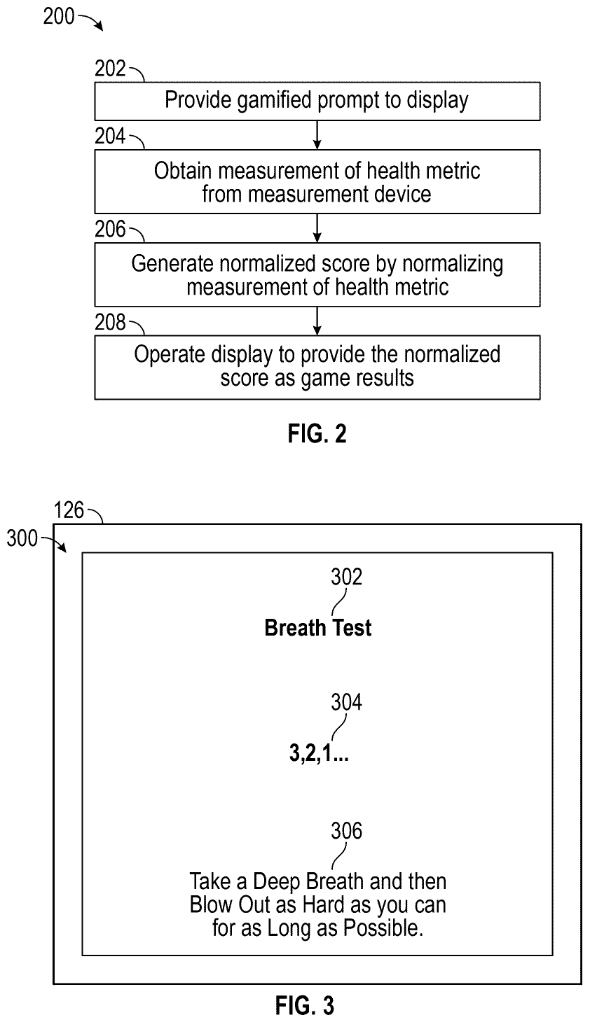

Referring now to FIG. 2, a flow diagram of a process 200 (e.g., a method) for providing a normalized game score of a measurement of a health metric includes steps 202-208, according to some embodiments. The process 200 may be performed in order to operate a display to provide the normalized game score as game results, according to some embodiments. In some embodiments, the process 200 is performed or otherwise executed using various components of the computing environment 100.

The process 200 includes providing a gamified prompt to a display (step 202), according to some embodiments. In some embodiments, step 202 is performed by the analysis system 110 (e.g., by the content manager 124 and the IO device 126). In some embodiments, step 202 includes providing information to a display (e.g., the IO device 126, a display of the measurement device 140) that corresponds to a measurement device (e.g., measurement device 140). The measurement device may be configured to obtain a first measurement of a health metric of a first user. For example, the measurement device may be a spirometer configured to obtain a volume measurement of a lung capacity of the user, a rate of exhalation, a rate of inhalation, a peak rate of exhalation or inhalation, etc.

The process 200 includes obtaining a measurement of a health metric from a measurement device (step 204), according to some embodiments. In some embodiments, step 202 prompts a user to perform an action at the measurement device (e.g., to exhale or inhale into the measurement device 140) such that the measurement of the health metric may be obtained. After the processing circuitry 114 has provided the information to the display, the display may then provide the information to a user of the measurement device. For example, the information may include instructions for the user to operate the measurement device in order to produce a measurement of a health metric of the user. In some embodiments, step 204 is performed by the processing circuitry 114 by receiving signals or data from the measurement device 140.

The process 200 includes generating a normalized score by normalizing the measurement of the health metric (step 206), according to some embodiments. In some embodiments, step 206 is performed by the processing circuitry 114 based on the measurement of the health metric obtained in step 204. Step 206 may include obtaining population or demographic data of the health metric for a population or demographic to which the user of the measurement of the health metric belongs. In some embodiments, step 206 includes identifying an average or mean value of the health metric for the population or the demographic to which the user belongs. In some embodiments, step 206 includes scaling or normalizing the health metric relative to the average or mean value of the health metric for the population or demographic. In some embodiments, step 206 includes identifying a percentile of the population or demographic to which the measurement of the health metric belongs. In some embodiments, the normalized score generated in step 206 is normalized or scaled according to a range of a scale (e.g., a game scale). For example, if the game scale ranges from 1 to 100 points, or from 1 to 9000 points, step 206 may include scaling the percentile of the measurement of the health metric relative to a scale factor.

In some embodiments, step 206 includes generating a normalized score for multiple different measurements of health metrics obtained from users of different populations. For example, the populations or demographics may be based on age, sex, weight, etc., or any other factors of the user. For example, a first measurement of the health metric obtained for a five year old child that performs the action at the measurement device may be normalized relative to data associated with a population of other five year old children. Step 206 may include performing a first normalization to identify a first normalized score for the five year old child relative to the data of the population of other five year old children. Step 206 may include performing a second normalization, scaling, or conversion to transform the first normalized score to a game scale (e.g., 1 to 9000 points). Further in this example, if a twenty-five year old man provides a second measurement of the health metric by performing the action at the measurement device, the second measurement may first be normalized relative to data of the population of other twenty-five year old men, and then normalized, scaled, or converted to the game scale such that the resulting normalized scores of the five year old child and the twenty-five year old man may be compared in a fair manner. In some embodiments, the results of the first normalization (e.g., the respective ranking or percentile in the population of the user) are used as the game score.

The process 200 includes operating a display to provide the normalized score(s) of step 206 as game results (e.g., in a gamified manner) (step 208), according to some embodiments. In some embodiments, step 208 is performed by the processing circuitry 114 and the IO device 126. In some embodiments, the information provided to the user via the display (e.g., in any of step 202 or step 208) may include content relating to a game (e.g., a computer game, a video game, a simulation, etc.) involving the first measurement of the health metric. The game may be any game that may be used to form a game objective (e.g., a game goal, etc.) as well as corresponding animation. The game may include such concepts as keeping an object in the air, moving an object a distance (e.g., a golf simulator), swimming a distance, collecting elements, or any other concept that include an objective. For example, in some embodiments, the processing circuitry 114 may provide the information relating to a gamified prompt to the display describing the game involving the measurement of the health metric including instructions for how a user may participate in the game. In some embodiments, the information may include a prompt for the first user to perform an action relating to the measurement deice in order to obtain the first measurement and/or in order to achieve a game objective of the game.

Referring to FIG. 3, the processing circuitry 114 may provide the information to the display as a user interface (e.g., a graphical user interface (GUI), graphical user interface 300 graphical user interface 600, etc.). In some embodiments, the user interface may include an actionable item. For example, in some embodiments, the actionable item could be a text field for the user to enter individual information corresponding to the user, multiple buttons for selecting a population corresponding to the user, a dial for adjusting a setting of the measurement device, a button for starting a test of the measurement device, to take other actions based on the user or the medical device, etc.

The graphical user interface 300 as shown in FIG. 3 may be generated by the content manager 124 and transmitted to the IO device 126 or the measurement device 140 to be displayed to the user of the measurement device 140. As illustrated, the graphical user interface 300 includes a health metric indication 302, a prompt 304, and instructions 306. The health metric indication 302 may indicate a test performed by the measurement device on the user to generate the measurement of the health metric of the user or an action to be performed by the user in order to obtain the measurement of the health metric. For example, the health metric indication 302 may include text indicating that the test that is being performed by the measurement device is a lung capacity test to generate a volume measurement of the lunges of the user. In some embodiments, the health metric indication 302 may be part of a gamified prompt indicating a game involving the measurement of the health metric. For example, the health metric indication 302 may indicate that a game involving the volume of the lungs of the user measured by a spirometer includes raising an object into the air to a height based on the volume of the lungs of the user. In some embodiments, the health metric indication 302 may include content associated with the test and/or the game involving the measurement of the health metric, such as illustrations of the measurement equipment and/or illustrations associated with the game. For example, in some embodiments, the health metric indication 302 may include an illustration of a helicopter that is associated with a game involving taking a measurement of a flow rate of an exhale of a user that includes keeping a helicopter in the air for a time period based on the flow rate of the exhale of the user.

The prompt 304 may indicate to the user that the test performed by the measurement device is ready to commence. In some embodiments, the prompt 304 may include a count down to indicate to the user when to start the test. For example, the prompt 304 may count down from three to zero to indicate to the user that the test starts when the count down reaches zero. In some embodiments, the prompt 304 may include content related to the game involving the measurement of the health metric. For example, the prompt 304 may include a game element of the game moving into a position to indicate to the user that the test that is being performed by the measurement device is ready to commence.

The instructions 306 may indicate to the user the process for completing the test performed by the measurement device. In some embodiments, the instructions 306 may include text instructions that detail the process for completing the test. For example, the instructions 306 may indicate that for a breath test, the user should breathe in and then blow out (e.g., “Take a deep breath and then blow out as hard as you can for as long as possible”). In some embodiments, the instructions 306 may include photographs, illustrations, or videos indicating to the user the process for completing the test performed by the measurement device. For example, the instructions 306 may include a plurality of photographs that detail the steps that the user should take to complete the test.

In some embodiments, the instructions 306 may relate to the game involving the measurement of the health metric. For example, the instructions 306 may include text instructions that detail the process for completing the test performed by the measurement device in language that relates to the game. The instructions 306 may include photographs, illustrations, or videos that relate to the game and indicate to the user the process for completing the test performed by the measurement device. For example, the instructions 306 may include a video that shows a character featured in the game walking through the process for completing the test.

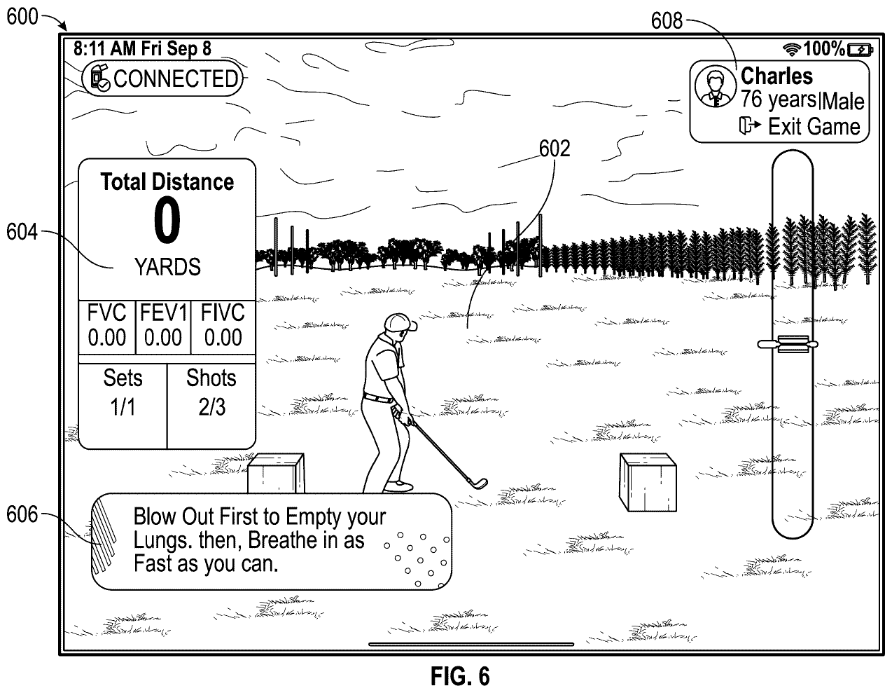

As another example, referring to FIG. 6, a graphical user interface 600 is shown, according to some embodiments. The graphical user interface 600 may be generated by the content manager 124 and transmitted to the IO device 126 or the measurement device 140 to be displayed to the user of the measurement device 140. As illustrated, the graphical user interface 600 includes a game environment 602, a game tracker 604, instructions 606, and personal information 608. The game environment 602 may include a background for a game involving the measurement of the health metric, elements associated with the game, and other components that may relate to the game. For example, the game environment 602 may include an illustration of a golf course and an illustration of a golfer for a game that relates to hitting a golf ball a distance based on a normalized score that is a normalization of a measurement of a health metric.

The game tracker 604 may include data relating to the game involving the measurement of the health metric. The data may include data related to the game or data related to the health metric. For example, the game tracker 604 may include a distance in yards and breath volume data for a game that relates to hitting a golf ball a distance based on a normalized score that is a normalization of a measurement of breath capacity. The instructions 606 may indicate to the user the process for completing the test performed by the measurement device. In some embodiments, the instructions 606 may include text instructions that detail the process for completing the test. For example, the instructions 606 may indicate that for a breath test, the user blow out and then breath in (e.g., “Blow out first to empty your lungs. Then, breathe in as fast as you can.”). The personal information 608 may include data relating to the user of the health device. For example, the personal information may include a name of the user, an age of the user, a gender of the user, or other relevant information associated with the user.

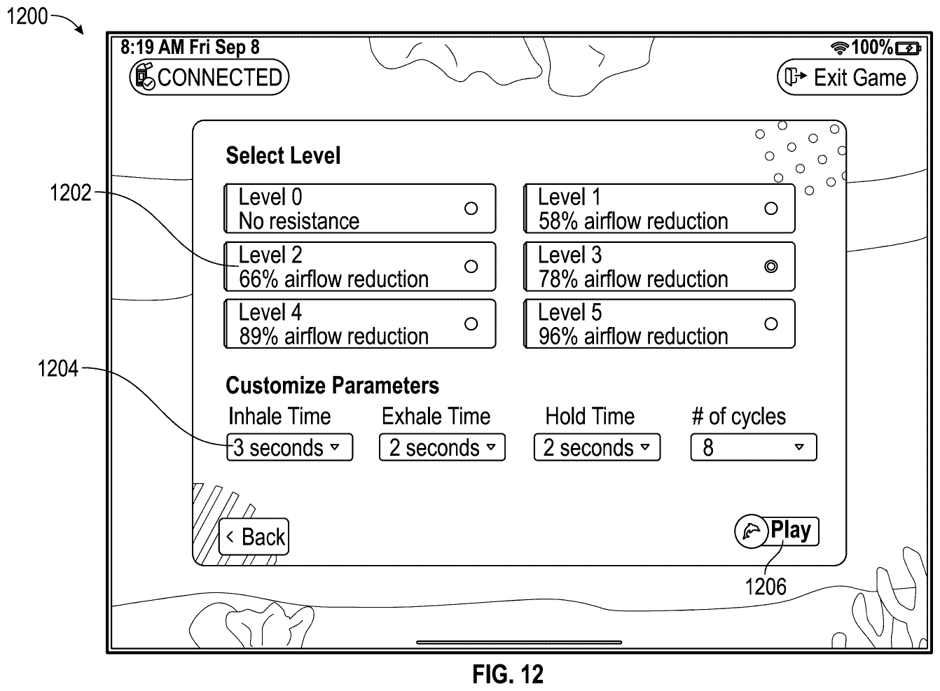

As another example, referring to FIG. 12, a graphical user interface 1200 is shown, according to some embodiments. In some embodiments, the graphical user interface 1200 is generated by the content manager 124 and transmitted to the IO device 126 or the measurement device 140 to be displayed to the user of the measurement device 140.

As illustrated the graphical user interface 1200 includes level selection elements 1202, parameter selection elements 1204, and a start element 1206. The level selection elements 1202 may include actionable elements that select a level of a game. For example, for a game that involves a normalized score that is a normalization of a measurement of lung health, the level selection elements 1202 may correspond to levels of the game. The levels of the game may be numbered such that the air flow restriction of the measurement device 140 increases to make the game more difficult as the number of the levels increase. The parameter selection elements 1204 may include actionable elements that modify parameters of a game. For example, for the game that involves a normalized score that is a normalization of a measurement of lung health, the parameter selection elements 1204 may change an inhale time, an exhale time, a hold time, a number of breathing cycles, etc. The parameter selection elements 1204 may be a text field for the user to enter the parameters of the game, a drop down menu for the user to select the parameters of the game, etc. The start element 1206 may be an actionable element that starts a game. For example, the selection of the start element 1206 may start the game that involves a normalized score that is a normalization of a measurement of a heath metric at a level corresponding to the level selection element 1202 that was selected and at parameters corresponding to the parameter selection elements 1204 that were selected.

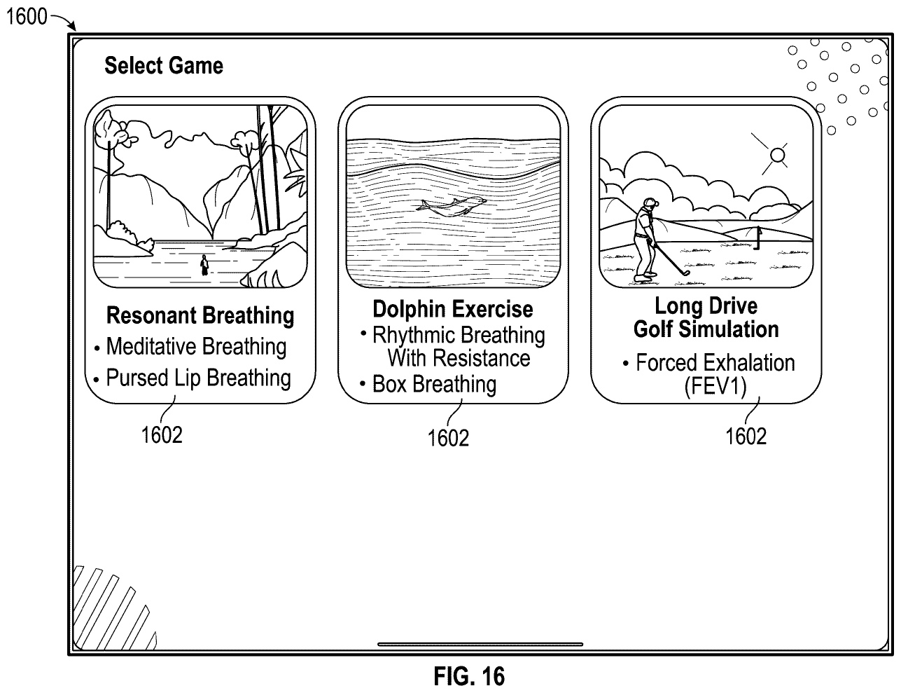

As another example, referring to FIG. 16, a graphical user interface 1600 is shown, according to some embodiments. The graphical user interface 1600 may be generated by the content manager 124 and transmitted to the IO device 126 or the measurement device 140 to be displayed to the user of the measurement device 140.

As illustrated, the graphical user interface 1600 includes a plurality of game icons 1602. Each of the plurality of game icons 1602 may include an actionable element that selects a different game associated with a normalized score that is a normalization of a measurement of a health metric. For example, for games involving a measurement of lung health, a first game may relate to meditative breathing, a second game may relate to rhythmic breathing with resistance, and a third game may relate to forced exhalation.

Once the display has been provided with the information, the processing circuitry 114 receives the first measurement of the health metric (e.g., metric, etc.) of the first user from the measurement device, at step 204. Additionally, the first user may belong to a first population of individuals (e.g., a first demographic, a first age group, etc.). The health metric may relate to lung health, muscle strength, flexibility, weight, blood pressure, or any other health attribute that may be measured. The first measurement may relate to a weight, a distance, a time, a pressure, or any other measurement that may be used to quantify the health metric. For example, in some embodiments, the information provided by the processing circuitry 114 may be related to blowing into a spirometer to generate a measurement of lung health. After receiving the information corresponding to the spirometer from the display, the user may blow into the spirometer to generate the measurement of lung health which may then be received by the processing circuit. The measurement of lung health may include a time that the user takes to fully exhale, a volume of air that the user blew into the spirometer, a maximum flow rate of air that the user blew into the spirometer, or any other measurements that may be taken by the spirometer. As another example, after receiving the information corresponding to a grip strength device from the display, the user may grip the grip strength device. The grip strength device may generate a measurement of grip strength which may then be received by the processing circuit. The measurement of grip strength may include a force applied by the user, a pressure applied by the user, or any other measurement that may be taken by the grip strength device.

In some embodiments, the processing circuitry 114 is configured to receive the first measurement of the health metric of the first user from the measurement device in real time (e.g., while the measurement device is collecting the measurement, while the user is performing the test, etc.). For example, the processing circuitry 114 may continually receive a measurement of a flow rate that the user blows into a spirometer while the user is still blowing into the spirometer. In some embodiments, the processing circuitry 114 is configured to receive the measurement of the health metric of the user from the measurement device at specific intervals of time. For example, in some embodiments, the processing circuitry is configured to receive the measurement of the health metric of the user from the measurement device once every tenth of a second.

Once the processing circuitry 114 has received the first measurement of the health metric of the first user from the measurement device, the processing circuitry 114 may generate a first normalized score (e.g., a standardized score, a scaled score, etc.) by normalizing the first measurement of the health metric of the first user relative to a plurality of measurements of the health metric of the first population of individuals (e.g., the population of the first user), at step 206. The measurements of the health metric of the first population of individuals may be stored in memory (e.g., memory 118) of the processing circuitry 114 or the measurements may be received from a data source (e.g., the data source 170). For example, the user may belong to a population of individuals who are between the ages of forty and fifty. The processing circuitry may normalize the measurement of the health metric of the user relative to the measurements of the health metric belonging to the population of individuals who are between the ages of forty and fifty.

To normalize the first measurement of the health metric of the first user relative to the measurements of the health metric of the first population, the processing circuitry 114 may use statistical normalization techniques such as z-score normalization, min-max scaling, or other methods of normalizing a data point based on a group of data. For example, the processing circuitry 114 may calculate an average measurement of the measurements of the health metric of a population of the user and then calculate a z-score of the measurement of the health metric of the user by subtracting the average measurement from the measurement and then dividing the result by the standard deviation of the measurements of the health metric of the population. The resulting z-score is a normalized value that indicates how many standard deviations the measurement of the health metric of the user is away from the average measurement and may be compared with other normalized values from other populations of individuals. This allows for the measurements of the health metrics of users in different of the populations of individuals to be compared based on how many standard deviations each of the measurements are from the average measurement of each of the populations instead of comparing the measurements directly to each other.

In some embodiments, the processing circuitry 114 may generate and adjust the first normalized score in real time (e.g., while the measurement device is collecting the measurement). For example, after receiving the measurement of the health metric of the user from the measurement device in real time, the processing circuitry 114 may generate the first normalized score based on the measurement. Then at a later moment in time, the processing circuitry 114 may receive an updated measurement of the health metric. The processing circuitry 114 may then generate the first normalized score based on the updated measurement. At an even later moment in time, the processing circuitry 114 may receive a final measurement of the health metric. The processing circuitry 114 may then generate the first normalized score based on the updated measurement.

In some embodiments, the processing circuitry 114 may generate the first normalized score at a time from the start of the test by normalizing the first measurement of the health metric of the first user at the time from the start of the test. For example, the processing circuitry 114 may receive a volume measurement that the user blew into a spirometer at a time from the start of the test. The processing circuitry 114 may then normalize the volume measurement that the user blew into the spirometer over the time from the start of the test relative to the volume measurements that a population group blew into the spirometer over the time from the start of the test.

Once the processing circuitry 114 has generated the first normalized score, the processing circuitry 114 may operate the display to provide the first normalized score as game results, at step 208. The game results may include elements that are related to a game and that correspond to the first normalized score. In some embodiments, the game results may be a positive result (e.g., a win, a success, etc.), a negative result (e.g., a loss, a failure, etc.), progress toward a game objective (e.g., a game goal, etc.), or an indication of how the first normalized score compares to another value (e.g., how the first normalized score compares to a second normalized score, etc.). For example, the game objective could be to achieve a normalized score above the average normalized score of a population of individuals that includes a user and the progress towards the game objective could indicate how far the first normalized score of the user is away from the average normalized score. In some embodiments, the processing circuitry 114 may operate the display to provide a game result interface (e.g., GUI, graphical user interface 400, graphical user interface 500, etc.).

In some embodiments, the processing circuitry 114 may compare the first normalized score to a predetermined value to determine the game results. For example, the predetermined value could be an average value of the normalized scores of the individuals from all of the populations. As another example, the predetermined value could be a threshold score that a medical professional has determined to be associated with a threshold, where normalized scores that are above the threshold score (e.g., normalized scores that exceed the threshold score, etc.) are healthy and normalized scores that are below the threshold score are unhealthy. Then, if the first normalized score is above the threshold score, the game results may be positive and if the first normalized score is below the threshold score, the game results may be negative.

In some embodiments, the processing circuitry 114 may compare the first normalized score to a predetermined range to determine the game results. For example, the predetermined range could be a range of normalized scores of the individuals from all of the populations. The game results may correspond to where the first normalized score is located across the predetermined range. For example, a game may include a goal to travel as far of a distance as possible along a path. If the first normalized score is on the low end of the predetermined range the game results may be to travel a shorter distance along the path than if the first normalized score is on the high end of the predetermined range.

In some embodiments, the processing circuitry 114 may operate the display to provide a second normalized score with the first normalized score as game results. In some embodiments, the second normalized score was generated by normalizing a second measurement of the health metrics of a second user in a second population of individuals to measurements of individuals of the second population of individuals. The game results may then correspond to how the first normalized score and the second normalized score compare to the predetermined value discussed above, how the first normalized score and the second normalized score compare to the predetermined range discussed above, or the relationship between the first normalized score and the second normalized score. By comparing the first normalized score to the second normalized score, the game results may allow for the first measurement of the health metric of the first user and the second measurement of the health metric of the second user to be compared based on a difference between the relation between the first measurement and the measurements of the first population and the relation between the second measurement and the measurements of the second population instead of directly comparing the first measurement to the second measurement.

In some embodiments, the processing circuitry 114 may compare the first normalized score to the second normalized score to determine the game results. For example, a first user and a second user may compete using the measurement device. A first measurement of the first user may be normalized with measurements of a first population of the first user to generate a first normalized score and a second measurement of the second user may be normalized with the measurements of a second population of the second user to generate a second normalized score. The processing circuitry 114 may then provide game results to the display that indicate which of the first normalized score or the second normalized score is higher instead of indicating which of first measurement and the second measurement is higher.

In some embodiments, the processing circuitry 114 may operate the display to provide a historical normalized score with the first normalized score as the game results. In some embodiments, the historical normalized score was generated by normalizing a historical measurement (e.g., a measurement taken in the past, etc.) of the health metric of the first user to measurements of individuals of the first population of individuals. The game results may then correspond to how the first normalized score and the historical normalized score compare to the predetermined value discussed above, how the first normalized score and the historical normalized score compare to the predetermined range discussed above, or the relationship between the first normalized score and the historical normalized score. In some embodiments, the historical measurement may have been taken by the measurement device when the first user belonged to a third population of individuals (e.g., a different demographic of individuals, a younger age group of individuals, etc.). In this case, the historical normalized score may be generated by normalizing the historical measurement of the health metric of the first user to measurements of individuals of the third population of individuals. The game results may then correspond to how the first normalized score compares with the historical normalized score such that the first user may determine how the health metric of the first user is trending compared to the population of individuals of the first user over time.

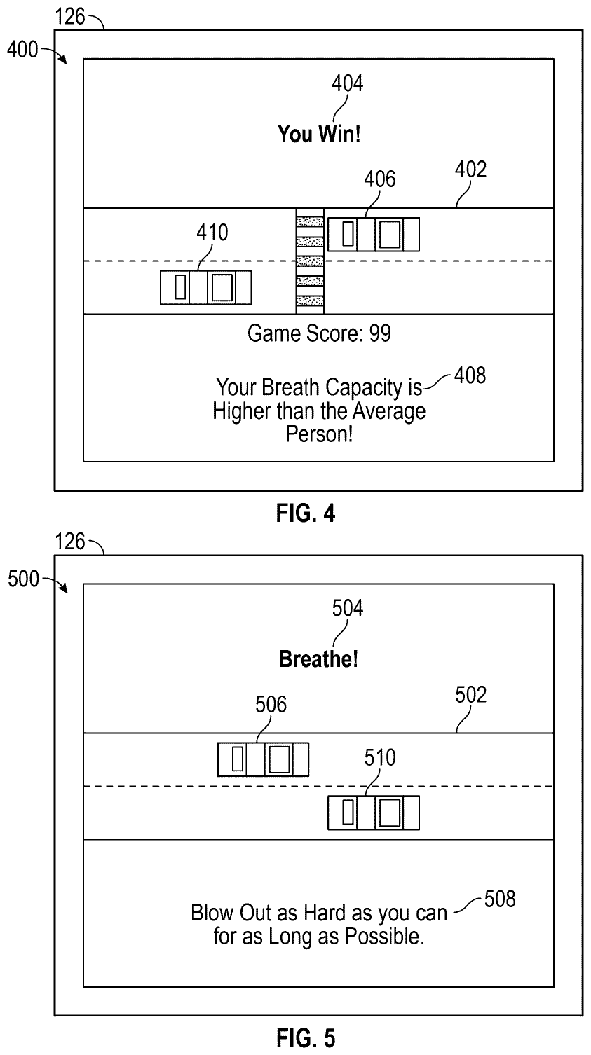

For example, referring to FIG. 4, a graphical user interface 400 is shown, according to some embodiments. In some embodiments, the graphical user interface 400 is generated by the content manager 124 and transmitted to the IO device 126 or the measurement device 140 to be displayed to the user of the measurement device 140.

As illustrated the graphical user interface 400 includes a game environment 402, a result indication 404, a user element 406, and a result explanation 408. The game environment 402 may include a background for a game involving the measurement of the health metric, elements associated with the game, and other components that may relate to the game. For example, the game environment 402 may include an illustration of a road and an illustration of a finish line for a game that relates to racing a vehicle based on a normalized score that is a normalization of a measurement of a health metric.

The result indication 404 may include an element corresponding to a game result. In some embodiments, the result indication 404 may include text indicating the game result. For example, the result indication 404 could include text stating that the user won the game (e.g., “You Win!”) to indicate that the game result is positive, text stating that the user lost the game (e.g., “You Lose!”) to indicate that the game result is negative, or text stating other results of the game. In some embodiments, the result indication 404 may include an illustration indicating the game result. For example, the result indication 404 may include an illustration of the trophy to indicate that that the game result is positive.

The user element 406 may be an element that indicates the game result of the user associated with a measurement of a health metric. The user element 406 may be an illustration of an element associated with the game. For example, the user element 406 may be an illustration of a vehicle for the game that relates to racing a vehicle based on a normalized score that is a normalization of a measurement of a health metric. In some embodiments, the user element 406 may be moved across the graphical user interface 400 based on the game results. For example, the illustration of the vehicle may move across a finish line included in the game environment 402 to indicate that the game result is positive, or the vehicle may not move across the finish line included in the game environment 402 to indicate that the game result is negative.

The result explanation 408 may include an explanation of the game result. In some embodiments, the result explanation 408 may include text explaining the game result. For example, if the game result is a comparison of a normalized score and an average normalized score, the result explanation 408 may include text stating that the normalized score is above the average normalized score (e.g., “Your breath capacity is higher than the person!”). In some embodiments, the result explanation 408 may include a visual explanation explaining the game results. For example, if the game result is a comparison of a normalized score and an average normalized score, the result explanation 408 may include a bar graph comparing the normalized score and the average normalized score.

In some embodiments, the graphical user interface 400 includes a comparison element 410. The comparison element 410 may represent a normalized score other than the first normalized score. The comparison element 410 may be an illustration of an element associated with the game, similar to the user element 406. For example, if the user element 406 is illustration of a vehicle for the game that relates to racing a vehicle based on a normalized score, the comparison element 410 may be a vehicle. In some embodiments, the comparison element 410 may differ from the user element 406 such that a user may tell the difference between the comparison element 410 and the user element 406. For example, the user element 406 and the comparison element 410 may be different colors, the user element 406 and the comparison element 410 may have different shapes, or the user element 406 and the comparison element 410 may have another visual difference. In some embodiments, the comparison element 410 may be moved across the graphical user interface 400 based on the game results similar to the user element 406.

In some embodiments, the comparison element 410 may represent the second normalized score of the second user. For example, the game may include a competition between a first user and a second user. The user element 406 may represent the game results of the first normalized score of the first user and the comparison element 410 may represent the game results of the second normalized score of the second user such that the first normalized score and the second normalized score are simultaneously displayed by the graphical user interface 400. In some embodiments, the game results may compare the first normalized score and the second normalized score to a predetermined value, to a predetermined range, or to each other. In other instances, the comparison element 410 may represent the historical normalized score of the first user. For example, the game may include a competition between a measurement of a first user and a historical measurement of the first user. The user element 406 may represent the game results of a first normalized score of the measurement the first user and the comparison element 410 may represent the game results of a historical normalized score of the historical measurement of the second user. In some embodiments, the game results may compare the first normalized score and the historical normalized score to a predetermined value, to a predetermined range, or to each other.

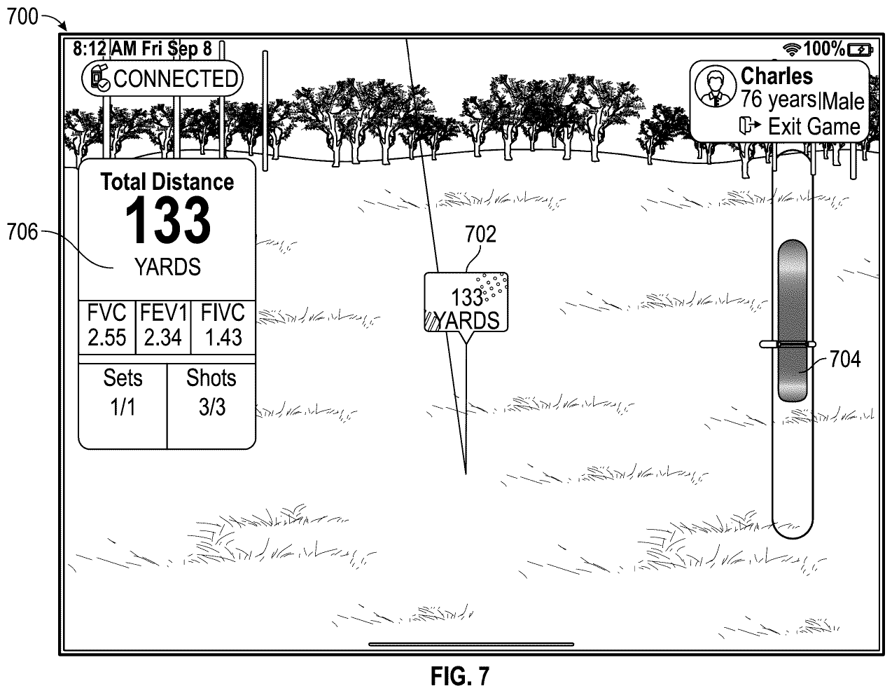

As another example, referring to FIG. 7, a graphical user interface 700 is shown, according to some embodiments. In some embodiments, the graphical user interface 700 is generated by the content manager 124 and transmitted to the IO device 126 or the measurement device 140 to be displayed to the user of the measurement device 140.

As illustrated the graphical user interface 700 includes a first result indication 702, a second result indication 704, and a result explanation 706. The first result indication 702 may include a first element corresponding to a game result. For example, for a game that involves hitting a golf ball a distance based on a normalized score that is a normalization of a measurement of a health metric, the first result indication 702 may be an illustration of a golf ball that flies the distance. The second result indication 704 may include a second element corresponding to a game result. For example, the second result indication 704 may include a bar that raises to a higher level based on a normalized score that is a normalization of a measurement of a health metric. In some embodiments, the second result indication 704 may change colors based on the game result. For example, a bar may change colors based on a normalized score (e.g., the bar may be green for a lower normalized score, the bar may be yellow for a medium normalized score, the bar may be red for a high normalized score, etc.)

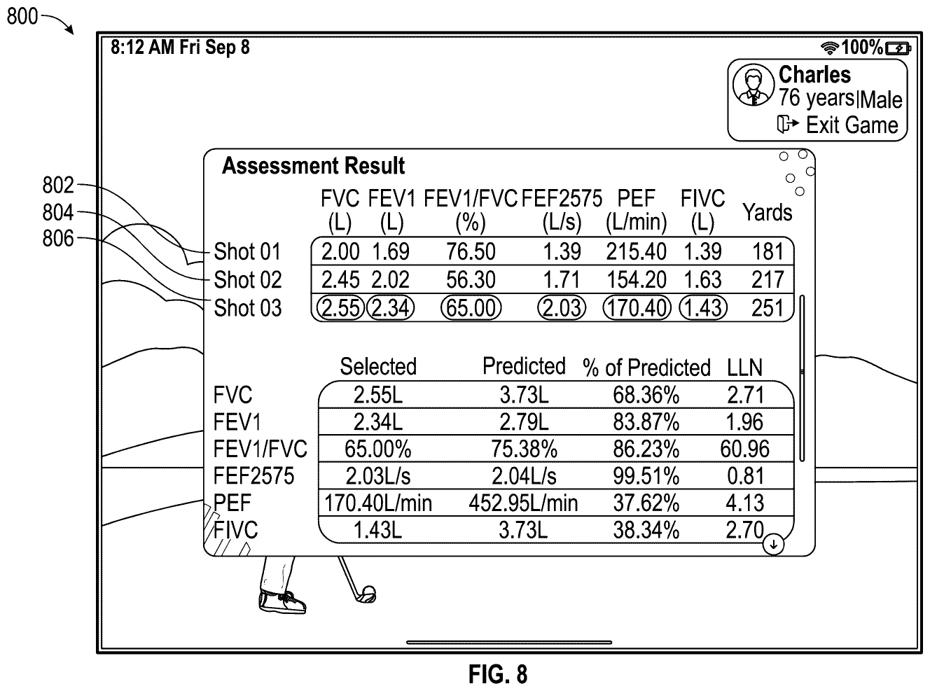

As another example, referring to FIG. 8, a graphical user interface 800 is shown, according to some embodiments. In some embodiments, the graphical user interface 800 is generated by the content manager 124 and transmitted to the IO device 126 or the measurement device 140 to be displayed to the user of the measurement device 140.

As illustrated the graphical user interface 800 includes a first game result 802, a second game result 804, and a third game result 806. The first game result 802 may include information associated with a first attempt of a game by a user. For example, for a game that involves hitting a golf ball a distance based on a normalized health metric corresponding to lung health, the first game result 802 may include at least one of a distance that a golf ball was hit or information relating to lung heath of a user. The second game result 804 may include information associated with a second attempt of the game by the user. The third game result 806 may include information associated with a third attempt of the game by the user.

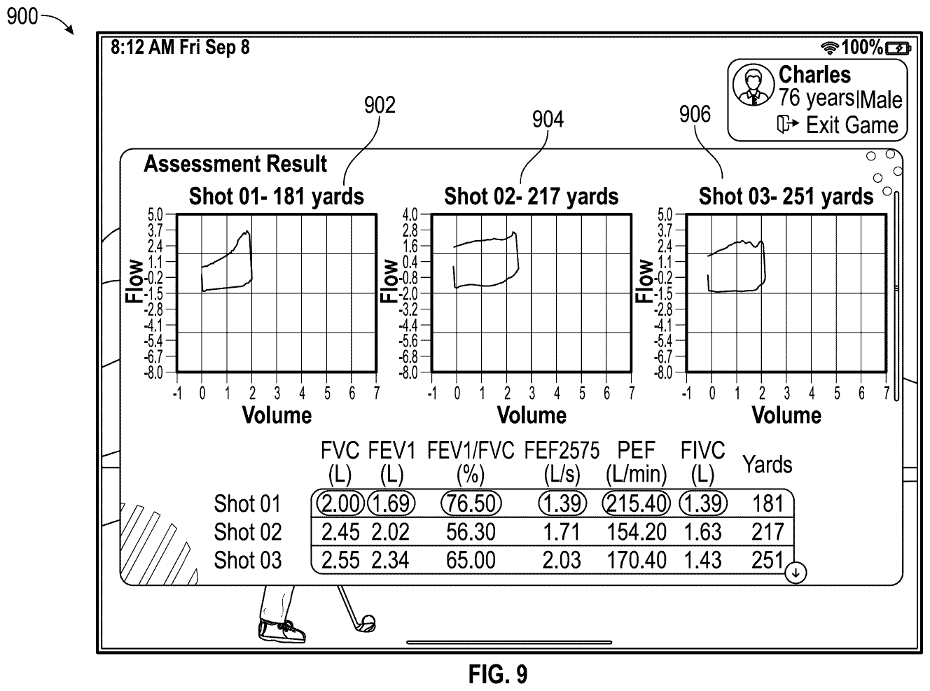

As another example, referring to FIG. 9, a graphical user interface 900 is shown, according to some embodiments. In some embodiments, the graphical user interface 900 is generated by the content manager 124 and transmitted to the IO device 126 or the measurement device 140 to be displayed to the user of the measurement device 140.

As illustrated the graphical user interface 900 includes a first graphical game result 902, a second graphical game result 904, and a third graphical game result 906. The first graphical game result 90 may include graphical information associated with a first attempt of a game by a user. For example, for a game that involves hitting a golf ball a distance based on a normalized health metric corresponding to lung health, the first graphical game result 902 may include at least one of a graph of a distance that a golf ball was hit or a graph relating to lung heath of a user. The second graphical game result 904 may include graphical information associated with a second attempt of the game by the user. The third graphical game result 906 may include graphical information associated with a third attempt of the game by the user.

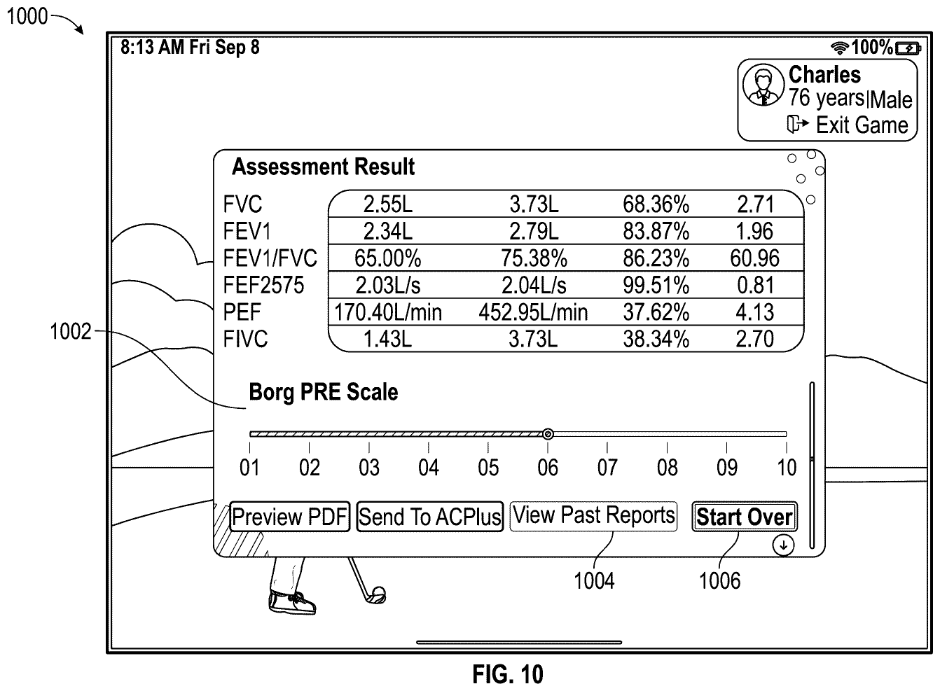

As another example, referring to FIG. 10, a graphical user interface 1000 is shown, according to some embodiments. In some embodiments, the graphical user interface 1000 is generated by the content manager 124 and transmitted to the IO device 126 or the measurement device 140 to be displayed to the user of the measurement device 140.

As illustrated the graphical user interface 1000 includes a normalized game score 1002 and a plurality of actionable elements 1004. The normalized game score 1002 may include an element corresponding to a normalized game score. For example, the normalized game score 1002 may be a sliding scale from one to ten that ranks a normalized score that is a normalization of a measurement of a health metric compared to a range of normalized scores. The plurality of actionable elements 1004 may be actionable items, prompts, or requests that the user may select to modify the graphical user interface 1000. The actionable elements 1004 may include a button for viewing a PDF of score results, a button for sending a normalized health metric to a health care system, a button for viewing past test results, a button for starting a test of the measurement device, etc.

In some embodiments, the processing circuitry 114 may operate the display to provide the first normalized score in real time as real time game results. For example, after receiving the first measurement of the health metric of the user from the measurement device in real time and generating the first normalized score based on the measurement, the processing circuitry 114 may operate the display to provide the first normalized score as real time game results. Then at a later moment of time after receiving the update measurement of the health metric and generating the first normalized score based on the updated measurement, the processing circuitry 114 may update the display to provide the first normalized score that is based on the updated measurement to the display as real time game results. In some embodiments, the processing circuitry 114 may update the display to provide the first normalized score that is based on the updated measurement at specific intervals of time. At an even later moment of time after receiving the final measurement of the health metric and generating the first normalized score based that is based on the final measurement, the processing circuitry 114 may update the display to provide the first normalized score that is based on the final measurement to the display as game results.

For example, referring to FIG. 5, a graphical user interface 500 is shown, according to some embodiments. In some embodiments, the graphical user interface 500 is generated by the content manager 124 and transmitted to the IO device 126 or the measurement device 140 to be displayed to the user of the measurement device 140.

As illustrated the graphical user interface 500 includes a game environment 502, an action prompt 504, a user element 506, and an instruction 508. The game environment 502 may include the elements discussed with regards to game environment 402 above. In some embodiments, the game environment 502 may adjust as the real time game results are updated by the processing circuit. For example, game environment could scroll to the left as the real time game results are updated to indicate movement of the user element 506 (e.g., progress of the user element 506, etc.).

The action prompt 504 may include an element corresponding to a game action. In some embodiments, the action prompt 504 may include text indicating the game action. For example, the action prompt 504 could include text stating an action that the user must take for the measuring device to determine the measurement (e.g., “Breath!” for a spirometer, etc.). In some embodiments, the action prompt 504 may include an illustration indicating the game action. For example, the action prompt 504 may include an illustration of a person taking the action that the user must take for the measuring device to determine the measurement (e.g., an illustration of a person breathing into a spirometer, etc.)

The user element 506 may include the elements discussed with regards to the user element 406 above. An aspect of the user element 506 may be updated as the first normalized score is updated in real time to indicate changes in the first normalized score. In some embodiments, the aspect of the user element 506 may be updated at specific intervals of time. In some embodiments, the aspect may be a size of the user element 506, a position of the user element 506, a color of the user element 506, or changes to other aspects of the user element 506. For example, the user element 506 may be an illustration of a vehicle for the game that relates to racing a vehicle based on a normalized score that and the position of the user element 506 may be updated as the normalized score is updated in real time.

The instruction 508 may include the elements discussed with regards to the instructions 306. In some embodiments, the instruction 508 may provide real time instructions to the user based on the first normalized scores determined in real time. For example, if a user is blowing into a spirometer at a flow rate that is not sufficient to take a measurement, the instruction 508 may include instructions to blow harder in order to be able to record a measurement.

In some embodiments, the graphical user interface 500 may include a comparison element 510. The comparison element 510 may include the elements discussed with regards to the comparison element 410. An aspect of the comparison element 510 may be updated as the first normalized score is updated in real time to indicate changes in the first normalized score. In some embodiments, the aspect of the comparison element 510 may be updated at the specific intervals of time that the user element 506 is updated. In some embodiments, the aspect may be a size of the comparison element 510, a position of the comparison element 510, a color of the comparison element 510, or changes to other aspects of the comparison element 510. The graphical user interface 500 may be operated to provide the user's game score as a quantified value (e.g., a normalized score in terms of game points), shown as “Game Score.” In some embodiments, the graphical user interface 500 includes a leaderboard illustrating different game scores of other individuals such as from different populations or demographics.

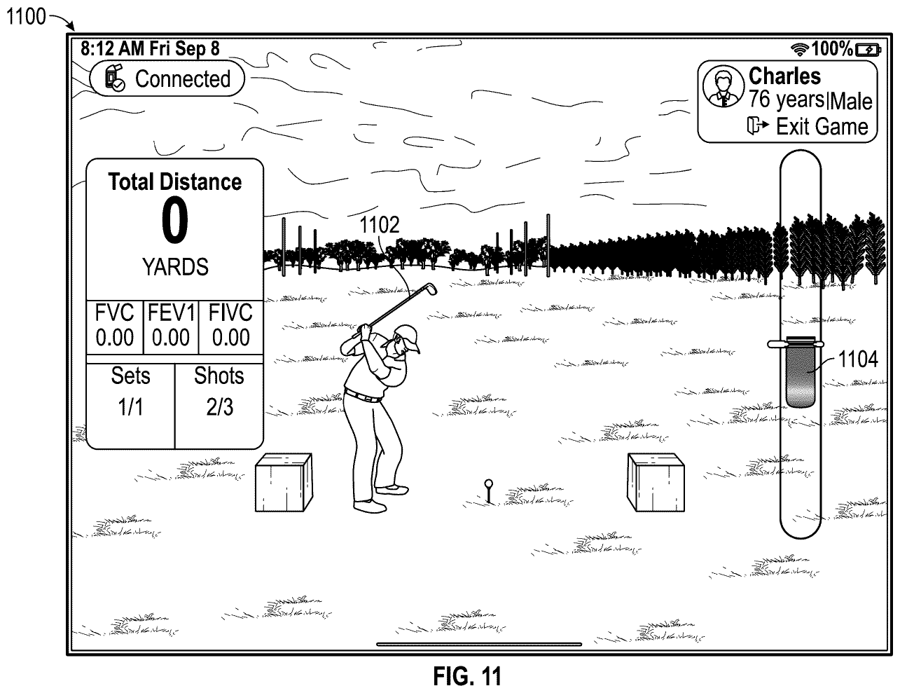

As another example, referring to FIG. 11, a graphical user interface 1100 is shown, according to some embodiments. In some embodiments, the graphical user interface 1100 is generated by the content manager 124 and transmitted to the IO device 126 or the measurement device 140 to be displayed to the user of the measurement device 140.

As illustrated the graphical user interface 1100 includes a user element 1102 and a result tracker 1104. The user element 1102 may be an element that indicates a game with a measurement of a health metric. The user element 1102 may be an illustration of an element associated with the game. For example, for the game that relates to hitting a golf ball a distance based on a normalized score that is a normalization of a measurement of a health metric, the user element 1102 may be an illustration of a golfer. The result tracker 1104 may include an element indicating the real time game results. An aspect of the result tracker 1104 may be updated as the first normalized score is updated in real time to indicate changes in the first normalized score. In some embodiments, the aspect may be a size of the result tracker 1104, a position of the result tracker 1104, a color of the result tracker 1104, or changes to other aspects of the result tracker 1104. For example, a bar may change colors based on a normalized health score (e.g., the bar may be green for a lower normalized health score, the bar may be yellow for a medium normalized health score, the bar may be red for a high normalized health score, etc.) and the color of the bar may change in real time.

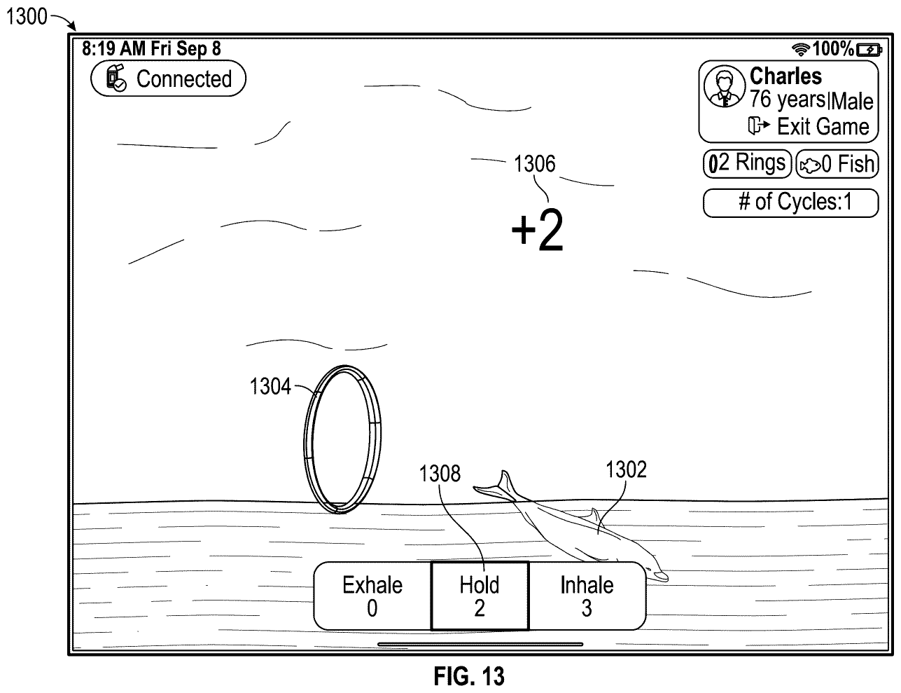

As another example, referring to FIG. 13, a graphical user interface 1300 is shown, according to some embodiments. In some embodiments, the graphical user interface 1300 is generated by the content manager 124 and transmitted to the IO device 126 or the measurement device 140 to be displayed to the user of the measurement device 140.

As illustrated the graphical user interface 1300 includes a user element 1302, a game target 1304, a score element 1306 and instructions 1308. The user element 1302 may be an illustration of an element associated with a game. For example, for a game that relates to a dolphin jumping out water and diving below the water based on a normalized score of a health metric relating to lung health, the user element 1302 may be an illustration of a dolphin for. The illustration of the dolphin may jump out of the water when a user exhales for the game relating to lung health and a height that the dolphin jumps out of the water may correspond to a property of the exhale of the user (e.g., a volume exhaled by the user, a velocity of the exhale of the user, etc.). Similarly, the illustration of the dolphin may dive deeper into the water when the user inhales for the game relating to lung health and a depth that the dolphin dives into the water may correspond to a property of the inhale of the user (e.g., a volume inhaled by the user, a velocity of the inhale of the user, etc.).

The game target 1304 may be an illustration of a goal associated with a game. For example, for a game that relates to a dolphin jumping out water based on a normalized score of a health metric relating to lung health, the game target 1304 may be an illustration of a hoop. The illustration of the hoop may be a target of the game such that when a property of an exhale of a user is above a threshold, a dolphin (e.g., the user element 1302) jumps through the hoop. The score element 1306 may indicate that a user has successfully completed an element of a game. For example, for a game that relates to a dolphin jumping out water based on a normalized score of a health metric relating to lung health, the score element 1306 may indicate that the user has successfully jumped the dolphin through a hoop and has scored points. The instructions 1308 may include the elements discussed with regards to the instructions 306. In some embodiments, the instruction 1308 may provide real time instructions to the user based on the normalized score determined in real time. The instructions 1308 may include a count down timer indicating to a user a time that the user should perform an action. For example, for a game that relates to a dolphin jumping out water based on a normalized score of a health metric relating to lung health, the instructions 1308 may include a first timer indicating a first time that a user should exhale, a second timer indicating a second time that the user should hold their breath, and a third timer indicating a third time that the user should inhale.

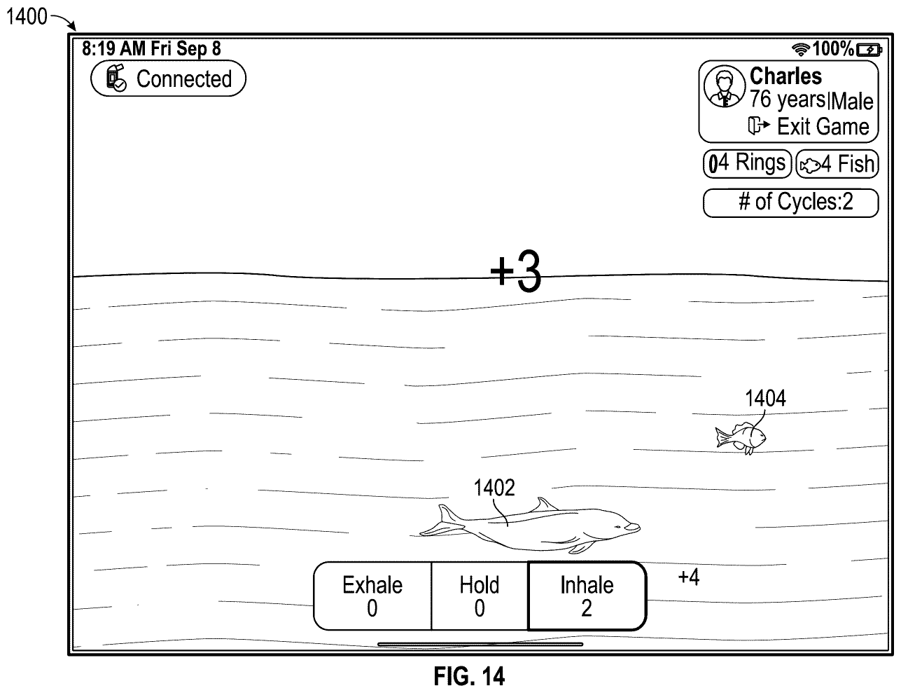

As another example, referring to FIG. 14, a graphical user interface 1400 is shown, according to some embodiments. In some embodiments, the graphical user interface 1400 is generated by the content manager 124 and transmitted to the IO device 126 or the measurement device 140 to be displayed to the user of the measurement device 140.

As illustrated the graphical user interface 1400 includes a user element 1402 and a game target 1404. The user element 1402 may be an illustration of an element associated with a game similar to the user element 1302 discussed above. The game target 1404 may be an illustration of a goal associated with a game similar to the game target 1304 discussed above. For example, for a game that relates to a dolphin jumping out water based on a normalized score of a health metric relating to lung health, the game target 1404 may be an illustration of a fish. The illustration of the fish may be a target of the game such that when a property of an inhale of a user is above a threshold, a dolphin (e.g., the user element 1402) eats the fish.

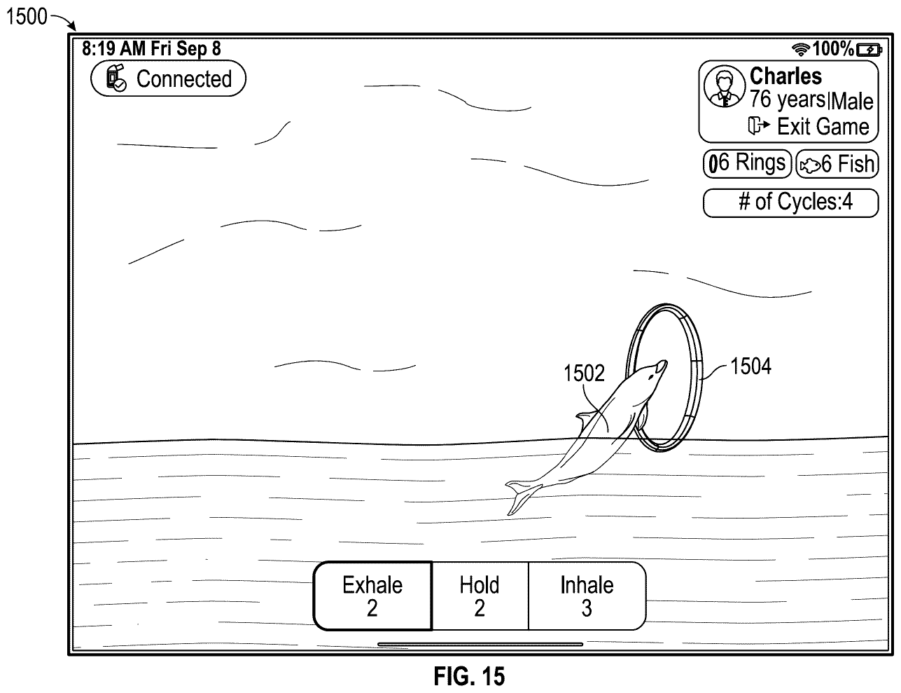

As another example, referring to FIG. 15, a graphical user interface 1500 is shown, according to some embodiments. In some embodiments, the graphical user interface 1500 is generated by the content manager 124 and transmitted to the IO device 126 or the measurement device 140 to be displayed to the user of the measurement device 140.

As illustrated the graphical user interface 1500 includes a user element 1502 and a game target 1504. The user element 1502 may be an illustration of an element associated with a game similar to the user element 1302 discussed above. The game target 1504 may be an illustration of a goal associated with a game similar to the game target 1304 discussed above. For example, for a game that relates to a dolphin jumping out water based on a normalized score of a health metric relating to lung health, the game target 1504 may be an illustration of a hoop. The illustration of the hoop may be a target of the game such that when a property of an inhale of a user is above a threshold, a dolphin (e.g., user element 1502) jumps through the hoop.

In some embodiments, various features of the graphical user interfaces described herein (e.g., graphical user interface 300, 400, 500, 600, 700, 800, 900, 1000, 1100, 1200, 1300, 1400, 1500, 1600) may be arranged differently and/or additional or alternative features may be included within the graphical user interfaces, without departing from the scope of the present disclosure.

It should be understood that no claim element herein is to be construed under the provisions of 35 U.S.C. § 112 (f), unless the element is expressly recited using the phrase “means for.”

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor), microprocessor, etc. In some embodiments, the one or more processors may be external to the apparatus, for example the one or more processors may be a remote processor (e.g., a cloud-based processor). Alternatively or additionally, the one or more processors may be internal and/or local to the apparatus. In this regard, a given circuit or components thereof may be disposed locally (e.g., as part of a local server, a local computing system) or remotely (e.g., as part of a remote server such as a cloud-based server). To that end, a “circuit” as described herein may include components that are distributed across one or more locations.

An exemplary system for implementing the overall system or portions of the embodiments might include general-purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. having computer-executable instructions. In some embodiments, the non-volatile media may take the form of ROM, flash memory (e.g., flash memory such as NAND, 3D NAND, NOR, 3D NOR), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example embodiments described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

Any foregoing references to currency or funds are intended to include fiat currencies, non-fiat currencies (e.g., precious metals), and math-based currencies (often referred to as cryptocurrencies). Examples of math-based currencies include Bitcoin, Litecoin, Dogecoin, and the like.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes, and omissions may be made in the design, operating conditions, and embodiment of the embodiments without departing from the scope of the present disclosure as expressed in the appended claims.

What is claimed is:

1. A system for scoring and displaying a health metric of a first user, the system comprising processing circuitry configured to:
provide a gamified prompt to the first user via a display;
obtain a first measurement of the health metric of the first user from a measurement device, the measurement device configured to obtain the first measurement of the health metric of the first user;
generate a first normalized score by normalizing the first measurement of the health metric of the first user relative to a plurality of measurements of the health metric of a first population of individuals, the first user being a member of the first population of individuals; and
provide the first normalized score to the display as game results.

2. The system of claim 1, wherein a second user is a member of a second population of individuals; and
wherein the processing circuitry is further configured to:
generate a second normalized score by normalizing a second measurement of the health metric of the second user relative to a plurality of measurements of the health metric of the second population of individuals; and
provide the second normalized score to the display as game results.

3. The system of claim 1, wherein the measurement device is a spirometer device, and the health metric is a metric indicating lung health.

4. The system of claim 1, wherein the gamified prompt is a prompt for the first user to perform an action in order to achieve a game objective, wherein the measurement device obtains the first measurement of the health metric of the first user as the first user performs the action.

5. The system of claim 4, wherein the processing circuitry is configured to provide the first normalized score to the display as a result of the action performed in order to achieve the game objective.

6. The system of claim 4, wherein the game results are positive when the first normalized score exceeds a threshold score, the threshold score corresponding with health metrics that are determined to be healthy.

7. The system of claim 1, wherein the game results include a game element that is operated to at least one of move at a speed based on the first normalized score, raise to a height based on the first normalized score, or move a distance based on the first normalized score.

8. The system of claim 1, wherein the first population of individuals is based on at least one of age or sex.

9. A method for scoring and displaying a health metric of a first user, the method comprising:
obtaining a first measurement of the health metric of a first user from a measurement device, the measurement device configured to obtain the first measurement of the health metric of the first user;
generating a first normalized score by normalizing the first measurement of the health metric of the first user relative to a plurality of measurements of the health metric of a first population of individuals, the first user being a member of the first population of individuals; and
providing the first normalized score as game results to the first user via a display.

10. The method of claim 9, further comprising:
obtaining a second measurement of the health metric of a second user from the measurement device;
generating a second normalized score by normalizing the second measurement of the health metric of the second user relative to a plurality of measurements of the health metric of a second population of individuals, the second user being a member of the second population of individuals; and
providing the second normalized score to the display as game results.

11. The method of claim 10, wherein the first normalized score and the second normalized score are simultaneously displayed on the display as game results.

12. The method of claim 10, wherein the first population of individuals and the second population of individuals are based on at least one of age or sex.

13. The method of claim 9, wherein the measurement device is a spirometer device, and the health metric is a metric indicating lung health.

14. The method of claim 9, wherein the game results are positive when the first normalized score exceeds a threshold score, the threshold score corresponding with health metrics that are determined to be healthy.

15. The method of claim 9, further comprising:
obtaining a second measurement of the health metric of the first user from the measurement device;
generating a second normalized score by normalizing the second measurement of the health metric of the first user relative to the plurality of measurements of the health metric of the first population of individuals; and
providing the second normalized score as game results to the display.

16. The method of claim 15, wherein the first normalized score and the second normalized score are simultaneously displayed on the display as game results.

17. A non-transitory computer-readable media having computer-executable instructions embodied therein that, when executed by at least one processor, causes the at least one processor to:
obtain a first measurement of a health metric of a first user from a measurement device, the measurement device configured to obtain the first measurement of the health metric of the first user;
obtain, from a database, a plurality of measurements of the health metric of a first population of individuals, the first user being a member of the first population of individuals;
generate a first normalized score by normalizing the first measurement of the health metric of the first user relative to the plurality of measurements of the health metric of the first population of individuals; and
provide the first normalized score to a display as game results.

18. The non-transitory computer-readable media of claim 17, wherein the instructions further cause the at least one processor to:
generate a second normalized score by normalizing a second measurement of the health metric of a second user relative to a plurality of measurements of the health metric of a second population of individuals, the second user being a member of the second population of individuals; and
provide the second normalized score to the display as game results.

19. The non-transitory computer-readable media of claim 18, wherein the instructions further cause the at least one processor to:
obtain a third measurement of the health metric of the first user from the measurement device;
generate a third normalized score by normalizing the third measurement of the health metric of the first user relative to the plurality of measurements of the health metric of the first population of individuals; and
provide the third normalized score as game results to the display.

20. The non-transitory computer-readable media of claim 17, wherein the measurement device is a spirometer device, and the health metric is a metric indicating lung health.
